(19) United States
(12) Patent Application Publication
(10) Pub. No.: US 2004/0082075 A1
(43) Pub. Date: Apr. 29, 2004

(54) Multi-step NMR for the identification and evaluation of biomolecule-compound interactions

(76) Inventor: Robert Powers, Westford, MA (US)

Correspondence Address: FISH & RICHARDSON P.C., 1425 K STREET, N.W., 11TH FLOOR, WASHINGTON, DC, 20005-3500, US

(21) Appl. No.: 10/280,899

(22) Filed: Oct. 25, 2002

(51) Int. Cl. G01N 24/00; G01N 33/68

(52) U.S. Cl. 436/173; 436/86; 435/7.1; 435/6

(57) ABSTRACT

Provided are methods of evaluating binding or a compound to a biomolecule. The methods utilize 1D NMR of the compound and 1D or 2D NMR of the biomolecule-compound mixture, along with 2D HSQC or TROSY methodology if a biomolecule-compound complex is formed. These methods are useful for evaluating biomolecule binding with more than one compound, e.g., a library of compounds, either individually or in mixtures. The methods are also useful for NMR evaluation of the effects of competition between a compound and a known binder such as an inhibitor of biomolecule activity.

### BACKGROUND

(1) Field of the Invention

The present invention generally relates to NMR methods for characterization of biomolecule-compound binding. More specifically, the invention provides multistep methods for identifying and characterizing binding between a biomolecule and a compound.

(2) Description of the Related Art

### REFERENCES CITED

Bax, A., Vuister, G. W., Grzesiek, S. & Delaglio, F. Measurement of homo- and heteronuclear J couplings from quantitative J correlation. Methods Enzymol. 239, 79-105 (1994).

Blundell, T. L. Structure-based drug design. Nature 384, Suppl., 23-26 (1996).

Chen, A. & Shapiro, M. J. NOE Pumping: A Novel NMR Technique for Identification of Compounds with Binding Affinity to Macromolecules. J. Am. Chem. Soc. 120, 10258-10259 (1998).

Clore, G. M. & Gronenborn, A. M. Structures of larger proteins, protein-ligand and protein-DNA complexes by multidimensional heteronuclear NMR. Protein Science 3, 372-390 (1994a).

Clore, G. M. & Gronenborn, A. M. Multidimensional heteronuclear nuclear magnetic resonance of proteins. Methods Enzymol. 239, 349-362 (1994b).

Cooke, R. M. Protein NMR extends into new fields of structural biology. Curr. Opin. Chem. Biol. 1, 359-364 (1997).

Dalvit, C. et al. Identification of compounds with binding affinity to proteins via magnetization transfer from bulk water. J. Biomol. NMR 18, 65-68 (2000).

Delaglio, F. et al. NMRPipe: A multidimensional spectral processing system based on UNIX pipes. J. Biomol. NMR 6, 277-293 (1995).

Fejzo, J. et al. The SHAPES strategy: an NMR-based approach for lead generation in drug discovery. Chem. Biol. 6, 755-769 (1999).

Fejzo, J. et al. The SHAPES strategy: an NMR-based approach for lead generation in drug discovery. Chem. Biol. 6, 755-769 (1999).

Gardner, K. H. & Kay, L. E. The use of 2H, 13C, 15N multidimensional NMR to study the structure and dynamics of proteins. Annu. Rev. Biophys. Biomol. Struct 27, 357-406 (1998).

Garrett, D. S., Powers, R., Gronenborn, A. M. & Clore, G. M. A common sense approach to peak picking in two-, three-, and four-dimensional spectra using automatic computer analysis of contour diagrams. J. Magn. Reson. 95, 214-20 (1991).

Gorse, D. & Lahana, R. Functional diversity of compound libraries. Curr. Opin. Chem. Biol. 4, 287-294 (2000).

Hajduk, P. J. et al. NMR-Based Discovery of Lead Inhibitors That Block DNA Binding of the Human Papillomavirus E2 Protein. J. Med. Chem. 40, 3144-3150 (1997a).

Hajduk, P. J. et al. Discovery of Potent Nonpeptide Inhibitors of Stromelysin using SAR by NMR. J. Am. Chem. Soc. 119, 5818-5827 (1997b).

Hajduk, P. J., Olejniczak, E. T. & Fesik, S. W. One-Dimensional Relaxation- and Diffusion-Edited NMR Methods for Screening Compounds That Bind to Macromolecules. J. Am. Chem. Soc. 119, 12257-12261 (1997c).

Hajduk, P. J. et al. High-Throughput Nuclear Magnetic Resonance-Based Screening. J. Med. Chem. 42, 2315-2317 (1999).

Hajduk, P. J. et al. NMR-Based Screening of Proteins Containing 13C-Labeled Methyl Groups. J. Am. Chem. Soc. 122, 7898-7904 (2000).

Kay, L. E., Keifer, P. & Saarinen, T. Pure absorption gradient enhanced heteronuclear single quantum correlation spectroscopy with improved sensitivity. J. Am. Chem. Soc. 114, 10663-5 (1992).

Kay, L. E. NMR methods for the study of protein structure and dynamics. Biochem. Cell Biol. 75, 1-15 (1997).

Konrat, R., Yang, D. & Kay, L. E. A 4D TROSY-based pulse scheme for correlating ¹HNᵢ, ¹⁵Nᵢ ¹³C_{iα}, ¹³Cᵢ₋₁ chemical shifts in high molecular weight, 15N, 13C, 2H labeled proteins. J. Biomol. NMR 15, 309-313 (1999).

Lewis, R. A., Pickett, S. D. & Clark, D. E. Computer-aided molecular diversity analysis and combinatorial library design. Rev. Comput. Chem 16, 1-51 (2000).

Lin, M., Shapiro, M. J. & Wareing, J. R. Diffusion-Edited NMR-Affinity NMR for Direct Observation of Molecular Interactions. J. Am. Chem. Soc. 119, 5249-5250 (1997a).

Lin, M., Shapiro, M. J. & Wareing, J. R. Screening Mixtures by Affinity NMR. J. Org. Chem. 62, 8930-8931 (1997b).

Lipinski, C. A. Drug-like properties and the causes of poor solubility and poor permeability. J. Pharmacol. Toxicol. Methods 44, 235-249 (2001).

Lipinski, C. A., Lombardo, F., Dominy, B. W. & Feeney, P. J. Experimental and computational approaches to estimate solubility and permeability in drug discovery and development settings. Adv. Drug Delivery Rev. 23, 3-25 (1997).

Mayer, M. & Meyer, B. Characterization of ligand binding by saturation transfer difference NMR spectroscopy. Angew. Chem., Int. Ed. 38, 1784-1788 (1999).

Meyer, B., Weimar, T. & Peters, T. Screening mixtures for biological activity by NMR. Eur. J. Biochem. 246, 705-709 (1997).

Moore, J. M. NMR techniques for characterization of ligand binding: utility for lead generation and optimization in drug discovery. Biopolymers 51, 221-243 (1999).

Moy, F. J. et al. MS/NMR: A Structure-Based Approach for Discovering Protein Ligands and for Drug Design by Coupling Size Exclusion Chromatography, Mass Spectrometry, and Nuclear Magnetic Resonance Spectroscopy. Anal. Chem. 73, 571-581 (2001).

Mulder, F. A. A., Ayed, A., Yang, D., Arrowsmith, C. H. & Kay, L. E. Assignment of ¹HN, ⁵N, ¹³Cα, ¹³CO and ¹³ Cβ resonances in a 67 kDa p53 dimer using 4D-TROSY NMR spectroscopy. J. Biomol. NMR 18, 173-176 (2000).

Ni, F. Recent developments in transferred NOE methods. Prog. Nucl. Magn. Reson. Spectrosc. 26, 517-606 (1994).

Nilakantan, R., Bauman, N. & Haraki, K. S. Database diversity assessment: new ideas, concepts, and tools. J. Comput.-Aided Mol. Des. 11, 447-452 (1997).

Nicholls, A., Sharp, K. & Honig, B. GRASP. PROTEINS, Structure, Function and Genetics 11, 281ff (1991).

Otting, G. Experimental NMR techniques for studies of protein-ligand interactions. Curr. Opin. Struct. Biol. 3, 760-8 (1993).

Pervushin, K., Riek, R., Wider, G. & Wüthrich, K. Attenuated T₂ relaxation by mutual cancellation of dipole-dipole coupling and chemical shift anisotropy indicates an avenue to NMR structures of very large biological macromolecules in solution. Proc. Natl. Acad. Sci. U.S.A. 94, 12366-12371 (1997).

Pervushin, K. V., Wider, G. & Wüthrich, K. Single transition-to-single transition polarization transfer (ST2-PT) in [¹⁵N, ¹H]-TROSY. J. Biomol. NMR 12, 345-348 (1998).

Pervushin, K. V., Wider, G., Riek, R. & Wüthrich, K. The 3D NOESY-[¹H,¹⁵N,¹H]-ZQ-TROSY NMR experiment with diagonal peak suppression. Proc. Natl. Acad. Sci. US.A. 96, 9607-9612 (1999).

Riek, R., Pervushin, K. & Wüthrich, K. TROSY and CRINEPT: NMR with large molecular and supramolecular structures in solution. Trends Biochem. Sci. 25, 462-468 (2000).

Roberts, G. C. K. Applications of NMR in drug discovery. Drug Discovery Today 5, 230-240 (2000).

Rossi, C., Donati, A. & Sansoni, M. R. Nuclear magnetic resonance as a tool for the identification of specific DNA-ligand interaction. Chem. Phys. Lett. 189, 278-80 (1992).

Schleucher, J. et al. A general enhancement scheme in heteronuclear multidimensional NMR employing pulsed field gradients. J. Biomol. NMR 4, 301-6 (1994).

Shan, X. et al. Assignment of ¹⁵N, ¹³Cα, ¹³Cβ, and HN resonances in an ¹⁵N, ¹³C, ²H labeled 64 kDa trp repressor-operator complex using triple-resonance NMR spectroscopy and 2H-decoupling. J. Am. Chem. Soc. 118, 6570-6579 (1996).

Shan, X., Gardner, K. H., Muhandiram, D. R., Kay, L. E. & Arrowsmith, C. H. Subunit-specific backbone NMR assignments of a 64 kDa trp repressor/DNA complex: a role for N-terminal residues in tandem binding. J. Biomol. NMR 11, 307-318 (1998).

Shapiro, M. J. & Wareing, J. R. High resolution NMR for screening ligand/protein binding. Curr. Opin. Drug Discovery Dev. 2, 396-400 (1999).

Shirakawa, M. et al. Interaction of the 1-cro repressor protein with operator DNA fragments monitored as to amide proton magnetic resonances. J. Mol. Struct 242, 355-66 (1991).

Shuker, S. B., Hajduk, P. J., Meadows, R. P. & Fesik, S. W. Discovering high-affinity ligands for proteins: SAR by NMR. Science 274, 1531-1534 (1996).

Spellmeyer, D. C. & Grootenhuis, P. D. J. Recent developments in molecular diversity. Computational approaches to combinatorial chemistry. Annu. Rep. Med. Chem 34, 287-296 (1999).

Vogtherr, M. & Peters, T. Application of NMR Based Binding Assays to Identify Key Hydroxy Groups for Intermolecular Recognition. J. Am. Chem. Soc. 122, 6093-6099 (2000).

Waldeck, A. R., Kuchel, P. W., Lennon, A. J. & Chapman, B. E. NMR diffusion measurements to characterize membrane transport and solute binding. Prog. Nucl. Magn. Reson. Spectrosc. 30, 39-68 (1997).

Whittle, P. J. & Blundell, T. L. Protein structure-based drug design. Annu. Rev. Biophys. Biomol. Struct 23, 349-75 (1994).

Willett, P. Chemoinformatics—similarity and diversity in chemical libraries. Curr. Opin. Biotechnol. 11, 85-88 (2000).

Wüthrich, K. NMR of proteins and nucleic acids, 292 (John Wiley & Sons, Inc., New York, 1986).

Xue, L. & Bajorath, J. Molecular descriptors in chemoinformatics, computational combinatorial chemistry, and virtual screening. Comb. Chem. High Throughput Screening 3, 363-372 (2000).

Yang, D. & Kay, L. E. Improved ¹HN-detected triple resonance TROSY-based experiments. J. Biomol. NMR 13, 3-10 (1999a).

Yang, D. & Kay, L. E. TROSY Triple-Resonance Four-Dimensional NMR Spectroscopy of a 46 ns Tumbling Protein. J. Am. Chem. Soc. 121, 2571-2575 (1999b).

NMR has recently been demonstrated to be a useful alternative to standard high-throughput screening (HTS) techniques to screen small molecules for their ability to bind biomolecule targets of interest (Shuker et al., 1996; Moore, 1999; Shapiro & Wareing, 1999; Roberts, 2000; Moy et al., 2001). This NMR screening information has an obvious utility in structure based drug discovery and design (Roberts, 2000; Wüthrich, 1986; Otting, 1993; Whittle & Blundell, 1994; Blundell, 1996). NMR, as a screening technique, has advantages over traditional biological based assays. A primary strength of NMR, compared to standard high-throughput screens, is its relative universal application for each new biomolecule target without incurring the need to redesign the screening protocol that is necessary with an HTS assay. Additionally, a typical HTS result will imply a biological response upon addition of the inhibitor, but as a result of the complexity of the screening protocol and the mechanism of monitoring a response, it is generally not feasible to infer a binding interaction between the ligand and the biomolecule of interest. Conversely, NMR provides direct evidence for binding between the ligand and biomolecule target through a variety of responses based on the type of NMR experiment (Wüthrich, 1986; Otting, 1993). Furthermore the information obtained from the NMR analysis can be used to identify the binding site and determine a co-structure of the biomolecule with the ligand (Roberts, 2000; Clore & Gronenborn, 1994a; Cooke, 1997; Kay, 1997).

Observation of a binding event by NMR may occur through changes in line-width and/or peak intensity (T₁ and T₂ relaxation changes) (Rossi et al., 1992; Hajduk et al., 1997c) change in the measured diffusion coefficient for the ligand (Lin et al., 1997a; Lin et al., 1997b; Waldeck et al., 1997), chemical shift perturbations for either the ligand or biomolecule (Wüthrich, 1986; Otting, 1993; Shirakawa et al., 1991), induced transferred NOE (trNOE) for the ligand (Ni, 1994; Vogtherr & Peters, 2000), a saturation transfer difference (STD) between either the biomolecule or bulk solvent (WaterLOGSY) to the ligand (Mayer & Meyer, 1999; Dalvit et al., 2000), appearance of new NOEs and/or intermolecular NOEs between the ligand and biomolecule (Clore & Gronenborn, 1994a; Chen & Shapiro, 1998). The different NMR techniques exhibit inherent strengths and weakness associated with amount of material required, the particular biomolecule, experiment time, ability to differentiate between non-specific and stoichiometric binders and ability to identify the ligand binding site, which determine their effectiveness and utility in an NMR based screen.

The “SAR by NMR” method, previously described by Hajduk et al., illustrates the utility of NMR to screen small molecules for their ability to bind biomolecules from observed chemical shift perturbations in 2D ¹H-¹⁵N HSQC spectra (Shuker, 1996; Hajduk et al., 1997a; 1997b; 1999) and 2D ¹H-¹³C HSQC spectra (Hajduk et al., 2000). In addition to determining if the small molecule binds the biomolecule, the observed chemical shift perturbations also allow for the identification of the binding site on the biomolecule surface. Nevertheless, the use of 2D HSQC NMR spectra as a screen has some significant obstacles that may limit its use in a high-throughput format. Mainly, the relatively low sensitivity of NMR requires significant quantities of isotope enriched biomolecule (>0.2 mM) and data acquisition time (>10 minutes) per sample that drastically impact the number of compounds that can be screened (Kay et al., 1992; Schleucher et al., 1994). Recently developed NMR cryoprobes and flow-through probes may provide some solutions to these issues since they may provide a 3-4 fold increase in sensitivity and a method for increased throughput, respectively (Shapiro & Wareing, 1999; Hajduk et al., 1999).

An alternative to 2D HSQC NMR spectra is the application of 2D transferred-NOE experiments to screen for ligands that bind biomolecules (Moore, 1999; Ni, 1994; Vogtherr & Peters, 2000). The 2D transferred-NOE experiment requires a minimal (μM) amount of unlabeled biomolecule, since the experiment requires a 20-30 fold excess of ligand relative to the biomolecule. Additionally, the trNOE experiment may provide information on the conformation of the ligand when bound to the biomolecule. The major disadvantage of the transferred-NOE experiment is the significant increase in the experiment acquisition time (>1 hr.) that severely limits the number of compounds that can be screened in reasonable amount of time. Also, the trNOE experiment does not identify the ligand binding site on the biomolecule.

1D NMR techniques, particularly relaxation measurements, diffusion-edited measurements, saturation transfer difference, NOE pumping, WaterLOGSY, and transferred NOEs (Moore, 1999; Shapiro & Wareing, 1999; Rossi et al., 1992; Hajdek et al, 1997a; Lin et al., 1997a; Lin et al., 1997b; Vogtherr & Peters, 2000; Mayer & Meyer, 2000; Dalvit et al., 2000; Chen & Shapiro, 1998; Meyer et al., 1997), and the utilization of a SHAPES compound library (Fejzo et al, 1999) minimize resource and sample requirements. These 1D NMR experiments eliminate the need for labeled biomolecule while simultaneously minimizing biomolecule quantities by requiring a lower concentration (nM-μM) and decreasing data acquisition times (<10 minutes). The SHAPES library uses a very small set of molecular scaffolds (˜150 compounds) to represent a larger library where hits are used for virtual screening of, e.g., a corporate compound collection. Again, the end result is to minimize both the sample requirement and experiment time. Unfortunately, these 1D NMR experiments do not provide information on the location of the binding site, may not be able to differentiate between non-specific and stoichiometric binders and the use of a small compound library reduces the chances of identifying an initial hit.

While NMR provides valuable approaches to screen compound libraries to identify ligands that bind a biomolecule target, each NMR technique incurs a significant disadvantage that severely limits the utility of the methodology in a high-throughput screening mode. The instant invention addresses those limitations.

### SUMMARY OF THE INVENTION

Accordingly, the inventor has discovered methods for evaluating biomolecule-ligand binding that do not have many of the disadvantages of previously developed methods.

Thus, the present invention is directed to methods of evaluating binding of a compound to a biomolecule. The methods comprise obtaining a 1D NMR spectra for the compound, and contacting the compound with the biomolecule to create a biomolecule-compound mixture. The methods also comprise evaluating whether the compound specifically binds to the biomolecule by obtaining 1D or 2D NMR spectra of the biomolecule-compound mixture, and analyzing that data to determine whether the compound binds to the biomolecule to form a biomolecule-compound complex. If a biomolecule-compound complex is formed, the biomolecule-compound complex is further analyzed by obtaining and evaluating additional NMR spectra for the biomolecule-compound complex using 2D HSQC NMR or 2D TROSY NMR methodology.

In other embodiments, the invention is directed to methods of determining binding of compounds in a library to a biomolecule. The methods comprise obtaining a 1D NMR spectra for each compound in the library and contacting compounds in the library with the biomolecule to create multiple biomolecule-compound mixtures. The methods also comprise evaluating whether each of the compounds specifically binds to the biomolecule by obtaining 1D or 2D NMR spectra of each biomolecule-compound mixture, and analyzing that data to identify compounds that bind to the biomolecule to form a biomolecule-compound complex. Each biomolecule-compound complex is then further analyzed by obtaining and evaluating additional NMR spectra for the biomolecule-compound complex using 2D HSQC NMR or 2D TROSY NMR methodology.

In related embodiments, the invention is directed to processes for selecting a compound that binds to a biomolecule. The processes comprise obtaining a library of compounds; determining binding of the compounds to the biomolecule using any of the methods described above; preparing a second library, the second library comprising structural analog compounds of a compound that forms a biomolecule-compound complex with the biomolecule; evaluating the analog compounds for desirable binding characteristics or an ability to affect an activity of the biomolecule; and selecting an evaluated analog compound that has desirable binding characteristics or affects an activity of the biomolecule.

### BRIEF DESCRIPTION OF THE DRAWINGS

FIG. 1 is a flow chart depicting some embodiments of the invention methods.

FIG. 2 shows examples of NMR spectra. Panel A shows NMR spectra for a compound with poor physical properties; panel B shows NMR spectra for a compound with good NMR behavior.

FIG. 3 shows examples of saturation transfer difference (STD) NMR spectra. Panel A shows NMR spectra for a compound that is capable of binding to a protein (top) and the compound complexed with the protein (bottom). Panel B shows NMR spectra for a compound that does not bind to a protein. Top—the compound alone; Bottom—the compound with the protein.

FIG. 4 shows NMR spectra of results of a 1D competition experiment. The left panel shows titration of a compound with increasing concentrations of HCV polymerase. The spectra of the free compound is shown in A); mixtures of the compound with 7.5 μM (B)), 15 μM (C)), and 30 μM (D)) HCV polymerase. The right panel shows results of competition between two compounds d and e. The spectra of the mixture of d and e is shown in A); B) shows the spectra of 60 μM e with 30 μM HCV polymerase; C) shows the spectra of addition of 60 μM d to B); D) shows the spectra of 60 μM d with 30 μM HCV polymerase; E) shows the spectra of addition of 60 μM e to D).

FIG. 5 shows results of a 2D TROSY analysis to probe the location of small molecule binding on a protein. In Panel A, spectral differences between the protein alone and in the presence of a compound can be discerned. In Panel B, residues in the active site required for activity are the dark colored residues in the center of the illustration. 2D TROSY allows residues corresponding to the peaks that change in the presence of the compound to be identified, shown as the dark residues in the center-right of the illustration.

FIG. 6 shows an analysis of the structure of a protein-ligand complex. Panel A shows the NMR spectra of a compound alone (bottom) and in the presence of 25 μM PTP-1B (top). Panel B shows the fit of the compound to an electron-density map in complex with PTP-1B.

FIG. 7 shows a method of extracting a ring-scaffold and the calculation of a structure-based hash-code.

### DETAILED DESCRIPTION OF THE INVENTION

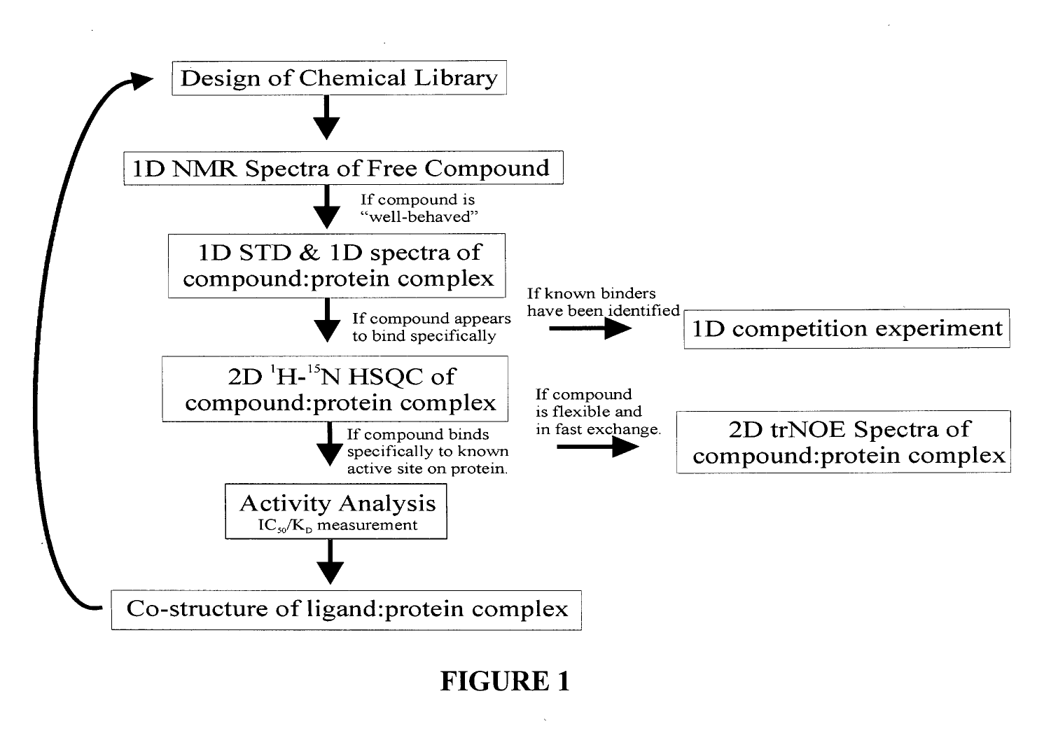

To circumvent these limitations of NMR in screening compound libraries to identify compounds that bind a biomolecule target, we have devised a multi-step NMR screen that emphasizes the strengths of each technique. The general flow of the multi-step NMR protocol is illustrated in FIG. 1. An important feature of the multi-step NMR screen is its flexibility and adaptability based on the particulars of the biomolecule system. There is an abundance of information obtainable from executing the entirety of the screen, but valuable information can be obtained from running only parts of the screen and/or substituting other components applicable to the biomolecule target under study. Therefore, the present invention is directed to the entire screen as well as novel parts of the screen.

In some embodiments, the invention is directed to methods of evaluating binding of a compound to a biomolecule. The methods comprise obtaining a 1D NMR spectra for the compound; contacting the compound with the biomolecule to create a biomolecule-compound mixture; and evaluating whether the compound specifically binds to the biomolecule by obtaining 1D or 2D NMR spectra of the biomolecule-compound mixture then analyzing the data in the evaluation step to determine whether the compound binds to the biomolecule to form a biomolecule-compound complex. If a biomolecule-compound complex is formed, the methods further comprise analyzing the biomolecule-compound complex by obtaining and evaluating additional NMR spectra for the biomolecule-compound complex using 2D HSQC NMR or 2D TROSY NMR methodology.

As used herein, a biomolecule is a large molecular weight (>1000 Dalton, more preferably greater than 2000 Dalton, even more preferably greater than 5000 Dalton) biological molecule. While in preferred embodiments, the biomolecule comprises a polypeptide potion, such as a protein, glycoprotein, nucleoprotein or lipoprotein, the invention also encompasses other biomolecules such as a nucleic acid, a lipid or a carbohydrate. Non-naturally occurring mimetics of these, such as phosphorothioate nucleic acid mimetics, are also within the scope of the invention. As used herein, a polypeptide is an unbranched chain of at least 10 amino acids.

Examples of polypeptide biomolecules useful in the invention are enzymes, cytokines, transcription factors, structural proteins, viral proteins, and bacterial proteins.

A compound is a molecule smaller than the biomolecule that is evaluated in the invention methods for interaction with the biomolecule. The compound is preferably an organic compound less than about 1000 Dalton. However, the invention is not limited to any particular type of compound, and includes organic molecules, single atom ions, oligopeptides, oligosaccharides, lipids, oligonucleotides such as aptamers, or mimetics.

In some preferred embodiments, the compound is part of a chemical library of more than one, preferably more than 10, even more preferably more than 50, compounds that are all assessed for interaction with the biomolecule. There are numerous and equally acceptable approaches to designing the chemical library utilized in the multi-step NMR screen. The variety of possible compound library designs has been described at length in the scientific community (Xue & Bajorath, 2000; Lewis et al., 2000; Willett, 2000; Spellmeyer & Grootenhuis, 1999; Gorse & Lahana, 2000). In general, the source of the compounds may be as diverse as screening the available compounds in a corporate library or utilizing a focused library designed specifically for the biomolecule target of interest. Additionally, chemical libraries may be designed that focus on specific properties of the compounds themselves, such as utilizing compounds that have “drug-like” properties and structural diversity (Lipinski et al., 1997; Lipinski, 2001). Also, the source of the chemical library may originate from the results of a standard biological assay or high-throughput screen.

In addition, the invention methods are equally amenable to utilizing either single compounds and/or mixtures. In the case of mixtures, the necessary de-convolution step could occur at multiple locations in the scheme where the preferred usage would be to repeat the 1D STD step for each component of the mixture for an identified hit. Using the 1D STD step clearly reduces both biomolecule materials, negates the need for labeled biomolecule and significantly reduces experiment time. Alternatively, the de-convolution step can take place at the 2D HSQC or 2D TROSY phase of the screen to allow for simultaneous confirmation of hits with identification of the biomolecule binding site and verification of stoichiometric binding.

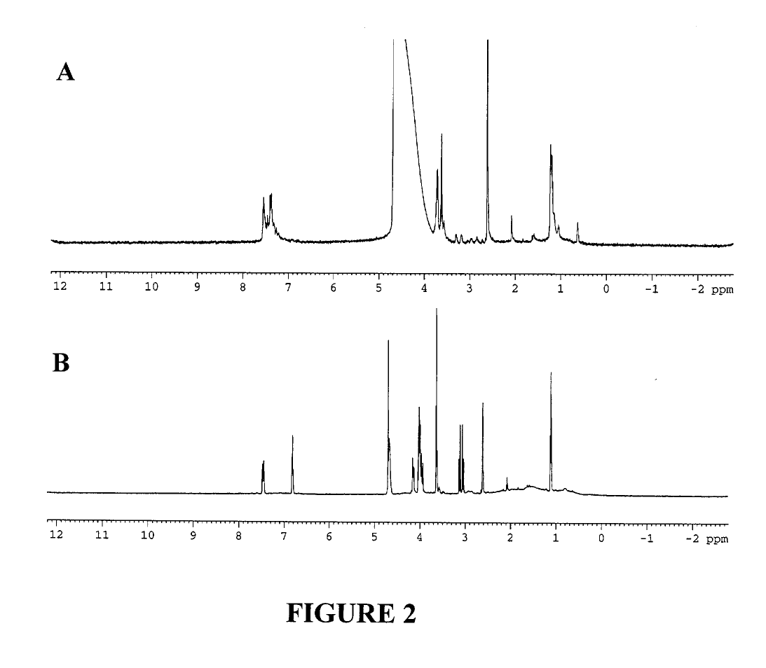

1D NMR Spectra of Free Compound A reference 1D NMR spectra for each individual compound is collected in a standard aqueous buffer and maintained as part of a database for future comparison to screening results to verify a proper hit (FIG. 2). The 1D NMR spectra for each compound only needs to be obtained the first time a compound is used as part of the multi-step NMR screen. In addition to providing a reference spectra, the 1D NMR spectra provides other critical information to evaluate the utility of the compound for screening and a structure-based design effort. The 1D NMR structure of the free compound indicates the relative aqueous solubility and stability of the compound, the compound's tendency to form high-molecular weight aggregates or micelle-like structures and, in addition, the accuracy of the structure. A compound that exhibits unusually broad-lines for a small-molecular weight compound suggestive of aggregation and/or micelle-like behavior is illustrated in FIG. 2.

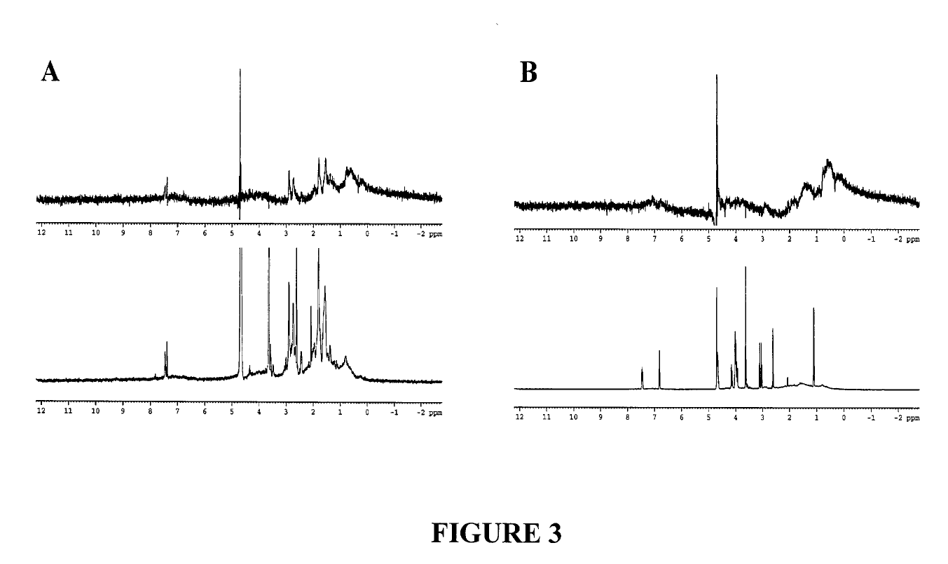

1D STD and 1D NMR Spectra of Biomolecule-Compound Complex. The first goal of the multi-step NMR screen is to identify compounds that bind the biomolecule target of interest while minimizing resources (biomolecule and instrument time). The 1D STD experiment addresses both these issues by utilizing unlabeled biomolecule samples as small as 1 nM with acquisition times on the order of minutes (Mayer & Meyer, 1999). Additionally, the screening step may be done as mixtures where the de-convolution of hits is accomplished by comparison of the resulting STD spectra with the database of free compound NMR spectra for proper identification of the compound that binds the target biomolecule. The basic principal of the STD approach is to observe binding between the biomolecule and the compound by the transfer of saturation from the biomolecule to the compound. The compound is preferably in large excess (˜20-30:1) relative to the biomolecule. Saturation occurs by selectively irradiating a region of the NMR spectrum that contains only biomolecule resonances, usually in the vicinity of 0.0 ppm. The NMR spectra is then collected by alternating between on- and off-resonance irradiation with appropriate phase cycling to record a difference spectrum. In cases where no binding takes place, the resulting NMR spectrum is a null. If binding does occur between the biomolecule and compound, then the resulting NMR spectrum would correspond to the spectrum of the free compound with some biomolecule background. An example of compounds that demonstrate both a positive STD (binding) and a negative STD (no binding) is illustrated in FIG. 3.

As used herein, “binding” or “interaction” does not imply any particular minimal affinity of the compound with the biomolecule. The affinity need only be sufficient to provide a biomolecule-compound complex of sufficient stability to obtain repeatable spectra.

A drawback of the STD experiment is its inability to distinguish between stoichiometric and non-specific binding. An initial step to identify highly non-specific binders is to simply collect a standard 1D NMR spectrum of the biomolecule-compound complex concurrent with the 1D STD experiment and to monitor line-width or relaxation difference (T₂). An alternative approach to the 1D STD methodology has been the measurement of a relaxation difference for the compound (Hajduk, et al., 1997a). NMR line-width is directly related to the intrinsic T₂ relaxation of the molecule, which in turn is directly correlated with the MW of the molecule. As a result of the large molecular weight difference between a biomolecule and a typical compound, there exists a large difference in the line-widths between the two. Therefore, a small molecule that binds a biomolecule will exhibit a significant increase in its NMR line-width, effectively broadening the spectra into the baseline. This effect only becomes pronounced enough to detect a difference when a significant percentage of the compound is bound to the compound, assuming a stoichiometric binder. At the high ratios of compound to biomolecule (˜20-30:1) generally used for the STD experiments, an observable change in line-width would only be detectable in cases where non-specific or multiple binding takes place between the compound and the biomolecule. From our extensive experience, there is a high-correlation between “good” behavior by a small-molecular weight compound in the NMR and future success in obtaining a co-structure. “Good” behavior is characterized by compounds that exhibit stoichiometric binding to the target biomolecule without solubility or aggregation issues and/or any observable detrimental impact on the biomolecule itself (precipitation, denaturation, etc.). Thus, the 1D STD and 1D NMR spectra of biomolecule-compound complex readily provides evidence of direct binding to the biomolecule while highlighting potential problematic compounds that may demonstrate high non-specific binding to the target biomolecule.

It is important to note that while the combination of the 1D STD and 1D NMR spectra represents the preferred first step in the multi-step NMR screen, the versatility of NMR presents numerous alternatives that may achieve similar results. Examples are relaxation measurements, diffusion-edited measurements, NOE pumping, Water LOGSY and transferred NOEs. Each experiment has its own strengths that may justify its use depending on the specifics of the system under study. Nevertheless, the fundamental underlying principal of the multi-step NMR screen is the utilization of a simple 1D or 2D NMR technique that would provide evidence of direct binding between the compound and the biomolecule while minimizing biomolecule and instrument resources and eliminating the need to use isotope labeled biomolecule.

Further evaluation of a biomolecule-compound interaction information is also useful using one or more NMR technique such as 1D STD, WaterLOGSY, transferred NOE, relaxation measurements, diffusion-edited measurements, NOE pumping, and/or a method that observes changes in chemical shifts, line width, peak height, NOE, a relaxation parameter (T₁, T_{1ρ}, T₂, etc.), and/or dynamic parameter (S², τ_{c}, Rₑₓ, etc.).

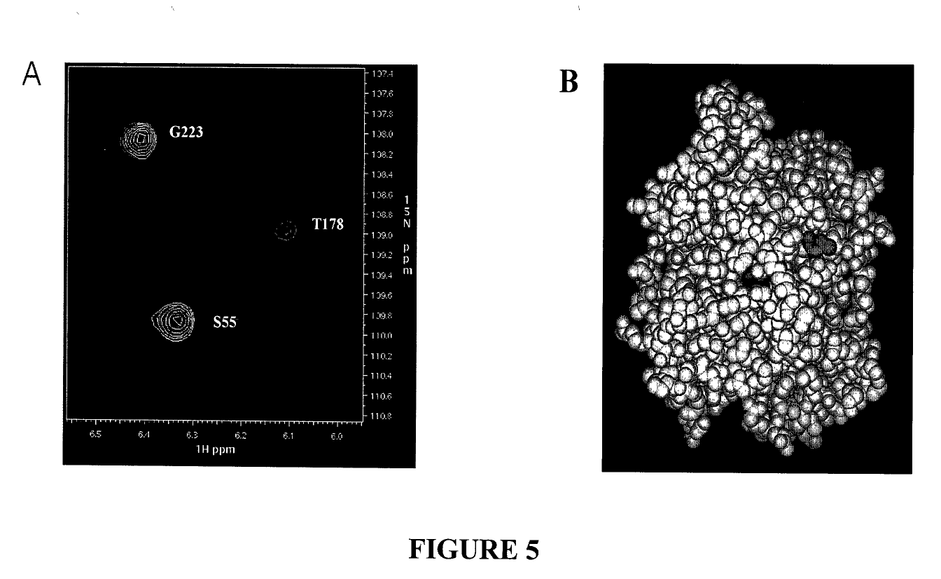

2D ¹H-¹⁵N HSQC Spectra of Biomolecule-Compound Complex The next step in the multi-step NMR assay is the evaluation of the hits from the 1D STD NMR by 2D ¹H-¹⁵N HSQC, 2D ¹H-¹³C HSQC NMR experiments, or for larger molecular-weight biomolecules (>25 kDa), the TROSY version of the 2D ¹H-¹⁵N HSQC experiment may be used (Riek et al., 2000) (FIG. 5). Since the NMR experiments are performed on identified hits, greater care can be taken to maximize the quality of the data and greater attention can be applied in the analysis of the data. This implies that a weak binding compound that may induce a minimal number of modest chemical shift perturbations has a less likelihood of being missed and that false positives resulting from pH or buffer changes may be eliminated.

In conjunction with previously determined NMR assignments and structure determination of the biomolecule target, it is a straightforward procedure to map the subunit residues (e.g., amino acids for a protein biomolecule or nucleotides for a DNA or RNA biomolecule) exhibiting chemical shift perturbations onto the biomolecule's molecular surface to define the binding site of an identified hit (FIG. 5). An observed clustering of residues in the same region of the biomolecule's surface also suggests a level of confidence that the compound is binding specifically to the biomolecule. Conversely, a random distribution or a complete lack of amino acids that incur a chemical shift perturbation is strongly suggestive of a non-specific binder. An automated approach for the analysis of the library of collected HSQC or TROSY spectra has been employed by using principal-component analysis (PCA) software, Tcl/Tk scripts written for the software program NMRWish (Delaglio et al., 1995) in combination with the GRASP (Nicholls et al., 1991) surface visualization software.

The NMR chemical shift perturbation analysis may be limited by the molecular weight of the biomolecule to about 35-45 kDa (Gardner & Kay, 1998; Pervushin et al., 1998), but chemical shift assignments have been obtained on a 64 kDa trp repressor/DNA complex (Shan et al., 1996; 1998) and recent developments in TROSY based experiments suggests a potentially higher molecular weight upper limit that may allow the collection of high-resolution NMR spectra of structures with molecular weights >100 kDa (Riek et al., 2000). NMR studies on “large” molecular weight biomolecule-compound complexes, where chemical shift assignments are not obtainable, may still provide direct evidence for binding and stoichiometry of low molecular weight molecules as well as the conformation of the bound compound. Alternatively, the use of ¹⁵N and/or ¹³C labeling of specific residue types may be used to partially map the interaction of the compound to the biomolecule. Additionally, chemical shift perturbations in the absence of the resonance assignments may still be useful in clustering hits based on distinct perturbation patterns. Also, comparison of chemical shift perturbation patterns between a known ligand with a defined binding site and a new binder may provide information on the binding site of the new compound. Additional information from 1D-competition experiments (as described above) may further define the interaction of the compound with the biomolecule target. Any combination of these approaches would provide information that would be invaluable for modeling the complex.

Essentially, the 2D HSQC NMR or 2D TROSY NMR data is complimentary to and expands the information content obtainable from the 1D STD NMR results. The 2D HSQC or 2D TROSY results further confirm the binding interaction of the compound with the biomolecule target while providing information on the binding site and further differentiating between non-specific and stoichiometric binders. Again, the nature of the biomolecule target being screened will dictate the specific details of the multi-step NMR screen that is performed and the utility of the 2D HSQC or 2D TROSY step.

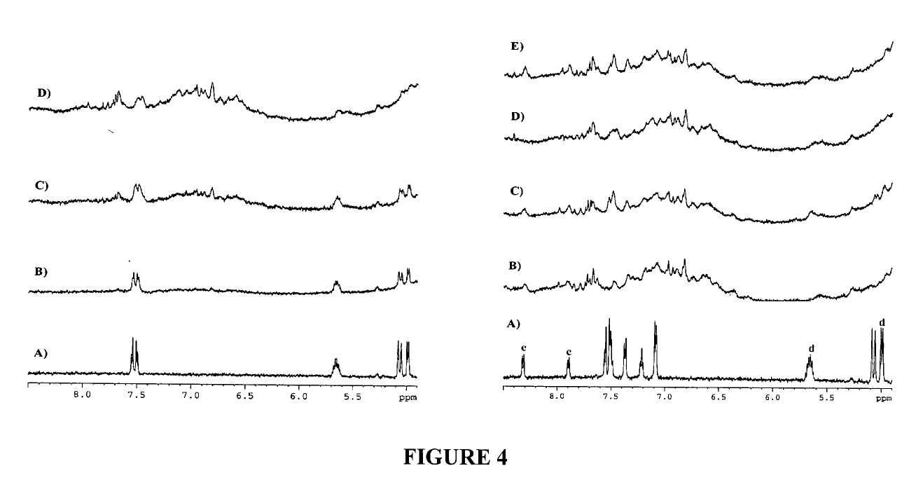

1D Competition Experiment. Depending on the specifics of the biomolecule target that is being screened, there may be value in determining if the hits from the previous binding analysis exhibit competitive binding to known substrates, ligands or other hits (FIG. 4). Again, the versatility of NMR permits several varieties, but fundamentally the concept is to follow a response from a 1D or 2D NMR experiment to monitor a potential loss in binding for a compound upon the addition of a known substrate, ligand or another hit. The preferred approach is to use 1D line-width changes of the compound upon addition of the biomolecule to verify binding. This is followed by addition of the known binder to the biomolecule-compound mixture to determine if the increase in line-width previously observed is now lost or reduced. Clearly, this result would suggest that the binding of the compound and known ligand is mutually exclusive and suggestive of a similar or overlapping binding site on the biomolecule. As an alternative to using known substrates or ligands, the same experiment may be done by comparison of different structural classes identified from the binding analysis. As an example, two compounds that have demonstrated binding to HCV polymerase were tested for competitive binding. In the left panel of FIG. 4 is the typical line-broadening titration for one of the compounds that establishes its binding to HCV polymerase. In the right panel of FIG. 4 are examples of co-titrations, where both HCV polymerase binders are present. In either case, the presence of the other compound does not reduce the binding of the HCV polymerase inhibitor as evident by a lack of a decrease in the NMR line-widths. These results were further confirmed by X-ray structures of the two inhibitors bound to HCV polymerase, where the compounds were shown to bind in distinct and distal locations in the protein structure.

Again, there is an inherent flexibility in the multi-step NMR screen as related to when the competition experiment is performed. It is equally valid to do the competition experiment after the 2D HSQC or 2D TROSY spectra or even after the activity validation step. Furthermore, the nature of the target may not even warrant doing a 1D-competition experiment.

Another important utilization of the 1D competition approach may be its application in the general multi-step NMR screening protocol to eliminate an unwanted class of compounds. Consider the situation where a known substrate, ligand or inhibitor exists for the biomolecule target of interest, but it is undesirable to identify compounds that bind in a similar manner. By having the known substrate, ligand, or inhibitor in molar excess relative to the compounds in the chemical library during the 1D STD NMR screening step, competitors to this known class of binders will be severely diminished. This will minimize wasted effort in follow-up experiments for inappropriate compounds. Additionally, this same approach may be used to explore alternative binding sites on the biomolecule with the end goal of chemically linking compounds that interact in the distinct binding sites or simply identifying an alternative interaction mode. Again, a compound that binds in a defined binding site is used during the 1D STD NMR screening step to direct the hits to a distinct binding site on the biomolecule.

Thus, in some preferred embodiments, a competition experiment is performed by evaluating the compound and/or biomolecule information using one or more of NMR technique such as 1D STD, WaterLOGSY, transferred NOE, relaxation measurements, diffusion-edited measurements, NOE pumping, and/or methods that observe changes in chemical shifts, line-width, peak height, NOEs, a relaxation parameter (T₁, T_{1ρ}, T₂, etc.) and/or a dynamic parameter (S², τ_{c} Rₑₓ, etc.); adding the known binder; then further evaluating the compound and/or biomolecule information using a technique selected from the group consisting of 1D STD, WaterLOGSY, a transferred NOE, a relaxation measurement, a diffusion-edited measurement, NOE pumping, and/or a method that observes changes in chemical shifts, line-width, peak height, NOEs, a relaxation parameter (T₁, T_{1ρ}, T₂, etc.) and/or a dynamic parameter (S², τ_{c}, Rₑₓ, etc.).

D trNOE Spectra of Biomolecule-Compound Complex. Another option of the multi-step NMR screen is to obtain 2D trNOE spectra of the biomolecule-compound complex with the goal of determining the bound conformation of the compound. This information can then be used in combination with the binding site identified from the 2D HSQC or 2D TROSY data to rapidly determine a model for the co-structure. The skilled artisan would understand that many factors will determine the utility of this step, including the conformational flexibility of the compound, availability of the biomolecule binding site, availability of the biomolecule structure and ease of determining a co-structure by NMR and X-ray.

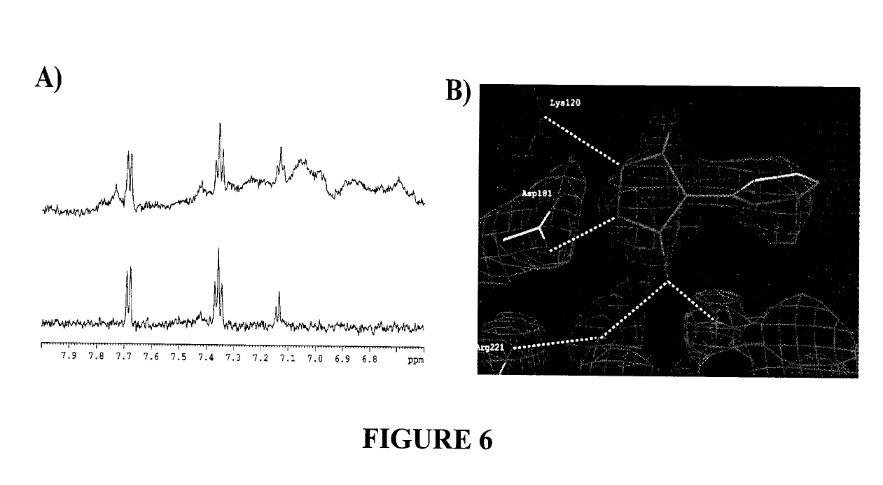

Inhibitor Activity, Structure Determination and Iterative Design. The confirmation by the multiple NMR experiments that these compounds bind specifically to the biomolecule target justify obtaining biological data correlating the observed biomolecule binding with a biological activity. Typically, IC₅₀ values are obtained for each ligand from a biological assay that provides an initial ranking of the effectiveness of the chemical leads. As a follow-up, K_{D} values can be obtained from NMR titration data or a variety of other analytical techniques (Otting, 1993; Ni, 1994). After verifying that the compounds bind to the biomolecule and effect the biomolecule activity, the structure of the biomolecule-compound complex is elucidated by NMR, X-ray, and/or modeling (FIG. 6). Finally, the assay protocol is amenable to an iterative approach where a library of structural analogs, based on the initial hits, can be used to further optimize the affinity and activity of the compound.

Thus, in some embodiments, the methods described above also comprise preparing a library of structural analogs of a compound that forms a biomolecule-compound complex with the biomolecule and evaluating the analogs for binding to the biomolecule or affecting activity of the biomolecule, using any of the methods described above, as appropriate. In preferred embodiments of these methods, an analysis of a biomolecule-compound complex is not performed with biomolecule-compound complexes where the biomolecule, compound, and/or biomolecule-compound complex has one or more undesirable NMR characteristic such as low solubility, tendency to form aggregates, compound instability, inaccurate structure, tendency to form micelle-like structures, and tendency to denature.

Preferred embodiments of the invention also include processes for selecting a compound that binds to a biomolecule. The processes comprise obtaining a library of compounds; determining binding of the compounds to the biomolecule using any of the above-described methods; preparing a second library, the second library comprising structural analogs of a compound that forms a biomolecule-compound complex with the biomolecule; evaluating the analogs for desirable binding characteristics or an ability to affect an activity of the biomolecule; and selecting an evaluated compound that has desirable binding characteristics or affects an activity of the biomolecule. Further iterations of the above-described obtaining a library of structural analogs if the selected compound; evaluating binding characteristics; selecting another evaluated compound; etc. could be performed to obtain further refinements of selected compounds.

Again, there is an inherent flexibility in the overall flow of the multi-step NMR screen that permits optimizing the approach for particulars of the biomolecule target that is being screened. This fact is particularly true for the activity analysis of the compounds, where the biological data may be obtained at any point during the screen. The preferred point for obtaining the activity information is after obtaining the 2D HSQC or 2D TROSY perturbation data as illustrated in FIG. 1. Nevertheless, depending on the availability of isotope enriched biomolecule it may be more valuable to obtain the activity data after the 1D STD and 1D spectra of the biomolecule-compound complex and prior to the 2D HSQC or 2D TROSY perturbation data. Also, the initial design of the chemical library may have been based on biological data that may negate the need for collecting activity data during the screen. Finally, the activity data may be obtained concurrently with any of the steps in the multi-step NMR screen.

Compounds that binds to a biomolecule, where the compound was selected using any of the above-described methods, are also within the scope of the invention.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### EXAMPLE 1

### Design of a Small Diverse Compound Library for NMR Screening

The Wyeth corporate compound collection was screened for compounds using the following criteria: molecular weight between 100 and 250, availability, calculated log p in the range of −10 to +3, no electrophiles, disulfides or thiols, i.e., amines and others. From the resulting compounds, a subset of compounds having unique ring-scaffolds was picked.

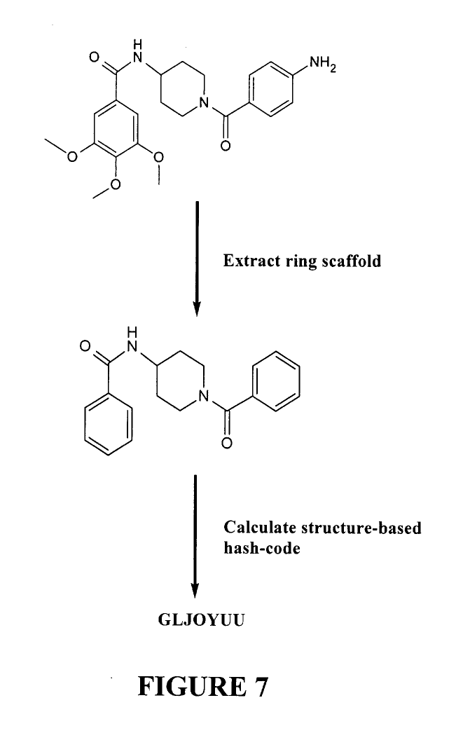

The ring-scaffold of a molecule is defined as the substructure remaining after all acyclic single-bonded appendages are removed. Note that acyclic chains connecting rings are part of the ring-scaffold. Also, note that the ring-scaffold as defined here preserves the atom-type information. FIG. 7 illustrates the definition with an example. In order to extract a set of compounds with unique ring-scaffolds, a structure-based 7-letter hash-code is calculated. The details of the calculation have been published elsewhere (Nilakantan et al., 1997).

### EXAMPLE 2

### PTP-1B Library

PTP-1B Screening. Using a small structurally-diverse library consisting of 825 compounds with aqueous solubility, 144 mixtures of 3-6 compounds were prepared at a concentration of 0.4 mM for each component in a buffer containing 20 mM TRIS, pH 7.5, 10 mM DTT in 90% D₂O with 10% DMSO. A 1D NMR spectrum for the individual compounds and the mixtures were collected to verify the utility of the samples. 20 μM of PTB1B was added to each mixture and both a 1D NMR spectrum and a 2D trNOE spectrum was collected. Analysis of the results indicated that 34 mixtures exhibited a positive response from the trNOE experiment. The trNOE experiments were re-collected for the 34 mixtures in the absence of PTP-1B to eliminate false positives. The remaining 29 mixtures were de-convoluted by preparing individual samples for each compound at a 0.4 mM concentration with 20 μM PTB1B and 10% DMSO in the same buffer described above. Standard 1D NMR spectra and a 1D STD spectra were collected on each sample. Out of the 174 de-convolution samples, 91 compounds exhibited a positive STD result, where 49 compounds yielded a strong STD signal and 1D NMR spectra did not suggest non-specific binding.

An additional 143 compounds identified from a high-throughput screen were analyzed individually by NMR using 250 μM compound, 10 μM PTP-1B, in the buffer described above in 10% DMSO. Standard 1D NMR spectra and a 1D STD spectra were collected on each sample. Forty nine compounds exhibited a positive STD result, where 24 compounds yielded a strong STD signal and the 1D NMR spectra was consistent with the reported structure. The remaining 25 hits demonstrated only weak STD signal and/or consistency between the 1D NMR spectra and the compounds structure was suspect.

Some of the compounds that exhibited the best inhibition results in the HTS assay were shown not to bind PTP-1B by STD. To verify that the 1D STD experiment was not inadvertently missing tight binders, 7 compounds were identified that had good inhibition in the HTS, no signal at all in the STD experiments, and good 1D NMR spectra (no obvious disagreement between spectrum and structure). NMR samples consisting of 80 μM of compound, 20 μM of PTP-1B in 2% deuterated DMSO in the buffer described above were examined for an increase in line-width for the compounds in the presence of PTP-1B. These results were compared to NMR spectra obtained for the compounds alone. None of the 7 compounds showed any line broadening in the presence of PTP-1B, suggesting that the STD results accurately identified all the compounds that bind PTP-1B.

Additionally, some compounds were evaluated for a binding interaction with PTP-1B by 1D line-broadening experiments. A 1D spectrum of each small molecule (60 μM) was collected in 20 mM Tris, 5 mM DTT, 1% DMSO, pH (measured) 7.5. 1D spectra were also collected upon addition of PTP-1B at the following concentrations: 60 μM small molecule/30 μM protein and 60 μM small molecule/15 μM protein. Compounds that exhibited significant change in their intrinsic NMR line-width upon the addition of PTP-1B would warrant further investigation.

Due to the high molecular weight of PTP-1B protein (35 kDa), the sensitivity and resolution achievable in typical NMR experiments (such as the 2D ¹⁵N HSQC) are poor. To improve the quality of spectra obtained for PTP-1B, two strategies were applied. First, the protein was extensively deuterated to remove the proton dipolar contribution to the relaxation of the NMR signals. Typically, a protein must be expressed in defined medium containing ¹⁵NH₄Cl or (15NH₄)₃SO₄ as the sole nitrogen source to achieve ¹⁵N labeling for 1H-15N heteronuclear correlation spectroscopy. For PTP-1B, ²H₂O was used as the solvent to achieve extensive deuteration in addition to 15N labeling. Second, transverse relaxation optimized spectroscopy (TROSY) was used to select the component of the signal with the best relaxation properties for detection (Pervushin et al., 1997). Note the cancellation of dipolar and CSA contributions to relaxation that gives rise to TROSY is more pronounced for higher molecular weight proteins that are deuterated and is more optimal at higher magnetic fields. The current data was collected at 600 MHz, but further improvements are expected at 800 MHz.

Expression of the protein in ²H₂O results in deuterium labeling at the backbone amide positions. However, ¹H is the most sensitive nucleus for biological NMR, so the deuterium must be exchanged back with ¹H. This is accomplished by incubating the PTP-1B in TBS, pH 7.5 supplemented with 5 mM DTT plus 1.6 M urea at room temperature for 1 hour. The protein is then dialyzed against two exchanges with 50 mM Tris, 150 mM NaCl, 15 mM DTT, uncorrected pH reading 7.5.

To examine the effect of compounds identified using the STD experiment on specific sites within the protein, 2D TROSY spectra were recorded on samples containing 400 ?M compound, 100 μM 2H ¹⁵N PTP-1B in 10% DMSO and 90% binding buffer with salt (20 mM deuterated Tris, uncorrected pH meter reading 7.5, 5 mM deuterated DTT, 150 mM NaCl, 10% 2H₂O). Spectra were recorded with 64 scans per increment, 116 complex points in ¹⁵N, and 1.2 seconds between scans for a total experiment time of 5 hours. Spectra were compared against a reference spectrum of protein alone using pcaView (Delaglio et al., 1995).

For 14 compounds identified from the STD screen, some minor changes in chemical shifts or peak intensity were observed for two compounds. Intensity changes were most noticeable for weaker peaks, where peaks near the active site (T178 and C215) get stronger suggesting that the compounds may bind near the active site and protect amide protons from exchange with solvent.

### EXAMPLE 3

### HCV Polymerase 1D Competition

HCV Polymerase 1D Competition experiment. The HCV polymerase construct used for the NMR binding studies has the 21 residues at the C-terminus deleted (Δ21). A 1D spectrum of each small molecule (60 μM) was collected in: 20 mM Tris (d11), 25 mM NaCl, 5 mM DTT (d), 5 mM MgCl₂, pH 7.2, and 5% DMSO. 1D spectra were also collected upon addition of Δ21 HCV polymerase at the following concentrations: 60 μM small molecule/30 μM protein and 60 μM small molecule/15 μM protein, and 60 μM small molecule/7.5 μM protein. 1D titrations with Δ21 HCV polymerase were collected individually for compounds (a) and (b). Then a titration with compound (a) was performed with HCV polymerase in the presence of molar excess of compound (b). The reverse titration was also performed.

### EXAMPLE 4

### PTP-1B Chemical Shift Assignments

Chemical shift assignments for the backbone of PTP-1B took advantage of ¹³C and ¹⁵N labeling to facilitate the assignment strategies based on through bond couplings (for reviews see Bax et al., 1994; Clore & Gronenborn, 1994b). As described above, to overcome challenges associated with PTP-1B's relatively high molecular weight (35 kDa), the protein was fully deuterated. Labeling was achieved by expressing the protein in defined medium containing ¹⁵NH4Cl as the sole nitrogen source and ²H, ¹³C glucose as the sole carbon source. Additionally, the solvent was ²H₂O, resulting in full deuteration of the protein. As described above, backbone amide ²H atoms were exchanged with ¹H by incubation in ¹H₂O-based buffer in the presence of 1.6 M urea.

Experiments to obtain the correlations necessary for assignments were implemented with TROSY to improve the relaxation properties of the backbone nuclei. Three pairs of experiments were recorded: TROSY-HNCA and TROSY-HN(CO) CA, TROSY-HNCO and TROSY-HN(CA)CO, and TROSY-HN(CA)CB, TROSY-HN(COCA)CB (Mulder et al., 2000; Konrat et al., 1999; Yang & Kay, 1999a; 1999b). Assignments were confirmed and extended using sequential HN-HN NOE crosspeaks, identified in an ¹⁵N separated NOESY spectrum, also implemented with TROSY and recorded with a 150 ms mixing time (Pervushin et al., 1999). Spectra were processed using nmrDraw and nmrPipe (Delaglio et al., 1995) and analyzed with PIPP (Garrett et al., 1991). Some connections between spin systems were identified using an in-house program, written in C.

Spectra of PTP-1B were recorded on a sample at 1 mM protein in 50 mM deuterated Tris, 100 mM NaCl, 15 mM deuterated DTT, uncorrected pH reading 7.5. Assignments are approximately 79% complete. Missing assignments are related to missing peaks in the 2D TROSY ¹H-¹⁵N map and weak peaks from that map that lack correlations in the 3D assignment spectra.

### EXAMPLE 5

### X-Ray Crystallographic Studies of PTP-1B-Inhibitor Complexes:

Cocrystallization of PTP-1B-Inhibitor Complexes. Cocrystals of PTP-1B (amino acids 1-298) with individual inhibtors were grown by hanging drop vapor diffusion at 4° C. A solution of ˜10 mg/mL PTP-1B in HEPES (25 mM, pH 7.5), NaCl (50 mM), DTT (3 mM), and EDTA (3 mM) was prepared, to which 1 mM inhibitor was added. For crystal growth, a 3 μL drop of complex solution was mixed with an equal volume of precipitating solution [PEG8000 (12-20%), MgCl₂ (0.1-0.3 M), HEPES (0.1 M, pH 7.5)] and equilibrated against 1 mL of the precipitating solution. The rod-like crystals (space group P3₁21) of PTP-1B-inhibitor complexes appeared within 7 days.

X-ray Diffraction Data Collection and Processing. A single crystal was transferred to a cryoprotectant solution, containing 25% glycerol in the precipitating solution. The crystal was then flash-frozen in the liquid nitrogen. Data were collected on an in-house X-ray generator with Raxis IV area detector or ALS at Berkeley National Laboratory with CCD detector and processed using the HKL software package.

Structure Determination and Refinement. A model of well refined PTP-1B crystal structure was used as starting model to perform the structure determination and refinement using CNS. 2Fo-Fc (or 3Fo-2Fc) and Fo-Fc maps were calculated. Solvent and inhibitor molecules were added accordingly based on the difference maps.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference in their entireties. The discussion of the references herein is intended merely to summarize the assertions made by the authors and no admission is made that any reference constitutes prior art. Applicants reserve the right to challenge the accuracy and pertinence of the cited references.

What is claimed is:

1. A method of evaluating binding of a compound to a biomolecule, the method comprising
a. obtaining a 1D NMR spectra for the compound;
b. contacting the compound with the biomolecule to create a biomolecule-compound mixture;
c. evaluating whether the compound specifically binds to the biomolecule by
i. obtaining 1D or 2D NMR spectra of the biomolecule-compound mixture; and
ii. analyzing the data in step i. to determine whether the compound binds to the biomolecule to form a biomolecule-compound complex; and
d. if a biomolecule-compound complex is formed, further analyzing the biomolecule-compound complex by obtaining and evaluating additional NMR spectra for the biomolecule-compound complex using 2D HSQC or TROSY NMR methodology.

2. The method of claim 1, wherein the biomolecule is a polypeptide.

3. The method of claim 2, wherein the polypeptide is an enzyme.

4. The method of claim 2, wherein the polypeptide is selected from the group consisting of a cytokine, a transcription factor, a structural protein, a viral protein, and a bacterial protein.

5. The method of claim 1, wherein the biomolecule is a nucleic acid.

6. The method of any one of claims 1-5, wherein more than one compound is in the biomolecule-compound mixture.

7. The method of any one of claims 1-6, wherein the spectra obtained in step c.i. is 1D NMR spectra.

8. The method of any one of claims 1-7, further comprising obtaining and evaluating biomolecule-compound interaction information using a technique selected from the group consisting of 1D STD, WaterLOGSY, a transferred NOE, a relaxation measurement, a diffusion-edited measurement, NOE pumping, and a method that observes changes in chemical shifts, line width, peak height, NOE, a relaxation parameter, and/or a dynamic parameter.

9. The method of any one of claims 1-8, wherein the spectra obtained in step d. is 2D ¹H-¹⁵N HSQC spectra.

10. The method of any one of claims 1-8, wherein the spectra obtained in step d. is 2D ¹H-¹³C HSQC spectra.

11. The method of any one of claims 1-8, wherein the spectra obtained in step d. is 2D TROSY spectra.

12. The method of claim 11, wherein the biomolecule is greater than 25 kDa.

13. The method of any one of claims 1-12, further comprising obtaining and evaluating 2D trNOE spectra of the biomolecule-compound complex.

14. The method of any one of claims 1-13, wherein a binding site of the compound to the biomolecule is further defined by mapping amino acid residues exhibiting chemical shift perturbations onto a molecular surface of the biomolecule.

15. The method of any one of claims 1-14, further comprising ¹⁵N and/or ¹³C labeling of a specific residue type.

16. The method of any one of claims 1-15, further comprising a competition NMR experiment with a second compound.

17. The method of claim 16, wherein the competition NMR experiment utilizes 1D NMR or 2D NMR.

18. The method of claim 16 or 17, wherein the second compound is a known binder of the biomolecule.

19. The method of claim 18, wherein the competition NMR experiment involves evaluating 1D line-width changes of the compound upon addition of the biomolecule, then addition of the known binder followed by a further evaluation of 1D line-width changes of the compound.

20. The method of claim 18 or 19, wherein the competition NMR experiment further comprises
evaluating the compound and/or biomolecule information using a technique selected from the group consisting of 1D STD, WaterLOGSY, a transferred NOE, a relaxation measurement, a diffusion-edited measurement, NOE pumping, and a method that observes changes in chemical shifts, line-width, peak height, NOEs, a relaxation parameter and/or a dynamic parameter;
adding the known binder; then
further evaluating the compound and/or biomolecule information using a technique selected from the group consisting of 1D STD, WaterLOGSY, a transferred NOE, a relaxation measurement, a diffusion-edited measurement, NOE pumping, and a method that observes changes in chemical shifts, line-width, peak height, NOEs, a relaxation parameter and/or a dynamic parameter.

21. The method of any one of claims 16-20, wherein the competition NMR experiment is performed after obtaining the 1D or 2D NMR spectra of step c.i.

22. The method of any one of claims 16-20, wherein the competition NMR experiment is performed after obtaining the 2D ¹H-¹⁵N HSQC or TROSY spectra of step d.

23. The method of any one of claims 17-22, wherein the second compound is an inhibitor of a function of the biomolecule.

24. The method of any one of claims 1-23, further comprising analyzing the effect of the compound on an activity of the biomolecule.

25. The method of claim 24, wherein the effect analyzed is inhibition of the activity of the biomolecule.

26. The method of any one of claims 1-25, further comprising analyzing the structure of a biomolecule-compound complex.

27. The method of any one of claims 1-26, further comprising
(A) preparing a library of structural analogs of a compound that forms a biomolecule-compound complex with the biomolecule; and
(B) evaluating the analogs for binding to the biomolecule or affecting activity of the biomolecule.

28. The method of any one of claims 1-27, wherein more than one biomolecule-compound mixture is evaluated.

29. A method of determining binding of compounds in a library to a biomolecule, the method comprising
a. obtaining a 1D NMR spectra for each compound in the library;
b. contacting compounds in the library with the biomolecule to create multiple biomolecule-compound mixtures;
c. evaluating whether each of the compounds specifically binds to the biomolecule by
i. obtaining 1D or 2D NMR spectra of each biomolecule-compound mixture; and
ii. analyzing the data in step i. to identify compounds that bind to the biomolecule to form a biomolecule-compound complex; and
d. further analyzing each biomolecule-compound complex by obtaining and evaluating additional NMR spectra for the biomolecule-compound complex using 2D HSQC or TROSY NMR methodology.

30. The method of claim 29, wherein the biomolecule is a polypeptide.

31. The method of claim 30, wherein the polypeptide is an enzyme.

32. The method of claim 30, wherein the polypeptide is selected from the group consisting of a cytokine, a transcription factor, a structural protein, a viral protein, and a bacterial protein.

33. The method of claim 29, wherein the biomolecule is a nucleic acid.

34. The method of any one of claims 29-33, wherein more than one compound is in the biomolecule-compound mixture.

35. The method of any one of claims 29-34, wherein the compounds of step b. do not include compounds in the library that have one or more undesirable characteristic identified by 1D NMR.

36. The method of claim 35, wherein the one or more undesirable 1D NMR characteristic is selected from the group consisting of low solubility, compound instability, inaccurate structure, tendency to form aggregates, tendency to form micelle-like structures, and tendency to denature the biomolecule.

37. The method of any one of claims 29-36, wherein the spectra obtained in step c.i. is 1D NMR spectra.

38. The method of any one of claims 29-37, further comprising obtaining and evaluating biomolecule-compound interaction information using a technique selected from the group consisting of 1D STD, WaterLOGSY, a transferred NOE, a relaxation measurement, a diffusion-edited measurement, NOE pumping, and a method that observes changes in chemical shifts, line-width, peak height, NOEs, a relaxation parameter and/or a dynamic parameter.

39. The method of any one of claims 29-38, wherein the analysis of step d. is not performed with biomolecule-compound complexes that have one or more undesirable biomolecule, compound, and/or biomolecule-compound complex NMR characteristic.

40. The method of claim 39, wherein the undesirable characteristic is selected from the group consisting of low solubility, tendency to form aggregates, compound instability, inaccurate structure, tendency to form micelle-like structures, and tendency to denature the biomolecule.

41. The method of any one of claims 29-40, wherein the information in step c.ii. is obtained from 1D STD.

42. The method of any one of claims 29-41, wherein the spectra obtained in step d. is 2D ¹H-¹⁵N HSQC spectra.

43. The method of any one of claims 29-41, wherein the spectra obtained in step d. is 2D ¹H-¹³C HSQC spectra.

44. The method of any one of claims 29-41, wherein the spectra obtained in step d. is 2D TROSY.

45. The method of claim 44, wherein the biomolecule is >25 kDa.

46. The method of any one of claims 29-45, further comprising obtaining and evaluating 2D trNOE spectra of the biomolecule-compound complex.

47. The method of any one of claims 29-46, wherein a binding site of the compound to the biomolecule is further defined by mapping amino acid residues exhibiting chemical shift perturbations onto a molecular surface of the biomolecule.

48. The method of any one of claims 29-47, further comprising ¹⁵N and/or ¹³C labeling of a specific residue type.

49. The method of any one of claims 29-48, further comprising a competition NMR experiment with a second compound.

50. The method of claim 49, wherein the competition NMR experiment utilizes 1D NMR or 2D NMR.

51. The method of claim 49 or 50, wherein the second compound is a known binder of the biomolecule.

52. The method of claim 51, wherein the competition NMR experiment involves evaluating 1D line-width changes of the compound upon addition of the biomolecule, then addition of the known binder followed by a further evaluation of 1D line-width changes of the compound.

53. The method of claim 51 or 52, wherein the competition NMR experiment further comprises
evaluating the compound and/or biomolecule information using a technique selected from the group consisting of 1D STD, WaterLOGSY, a transferred NOE, a relaxation measurement, a diffusion-edited measurement, NOE pumping, and a method that observes changes in chemical shifts, line-width, peak height, NOEs, a relaxation parameter and/or a dynamic parameter;
adding the known binder; then
further evaluating the compound and/or biomolecule information using a technique selected from the group consisting of 1D STD, WaterLOGSY, a transferred NOE, a relaxation measurement, a diffusion-edited measurement, NOE pumping, and a method that observes changes in chemical shifts, line-width, peak height, NOEs, a relaxation parameter and/or a dynamic parameter.

54. The method of any one of claims 49-52, wherein the competition NMR experiment is performed after obtaining the 1D or 2D NMR spectra of step c.i.

55. The method of any one of claims 49-52, wherein the competition NMR experiment is performed after obtaining the 2D ¹H-¹⁵N HSQC or TROSY spectra of step d.

56. The method of any one of claims 51-55, wherein the second compound is an inhibitor of a function of the biomolecule.

57. The method of any one of claims 29-56, further comprising analyzing the effect of the compound on an activity of the biomolecule.

58. The method of claim 57, wherein the effect analyzed is inhibition of the activity of the biomolecule.

59. The method of any one of claims 1-58, further comprising analyzing the structure of a biomolecule-compound complex.

60. The method of any one of claims 59, wherein the structure analyzed is a three-dimensional structure.

61. The method of claim 60, wherein the three-dimensional structure is analyzed using a method selected from the group consisting of molecular modeling, NMR spectroscopy, and X-ray crystallography.

62. The method of any one of claims 29-61, further comprising
(A) preparing a second library, the second library comprising structural analogs of a compound that forms a biomolecule-compound complex with the biomolecule; and
(B) evaluating the analogs for binding to the biomolecule or affecting activity of the biomolecule.

63. A process for selecting a compound that binds to a biomolecule, the process comprising
I. obtaining a library of compounds;
II. determining binding of the compounds to the biomolecule using the method of any one of claims 29-62;
III. preparing a second library, the second library comprising structural analog compounds of a compound that forms a biomolecule-compound complex with the biomolecule;
IV. evaluating the analog compounds for desirable binding characteristics or an ability to affect an activity of the biomolecule; and
V. selecting an analog compound evaluated in IV. that has desirable binding characteristics or affects an activity of the biomolecule.

64. The process of claim 63, wherein the biomolecule is a polypeptide.

65. The process of claim 64, wherein the polypeptide is an enzyme.

66. The process of claim 64, wherein the polypeptide is selected from the group consisting of a cytokine, a transcription factor, a structural protein, a viral protein, and a bacterial protein.

67. The process of claim 63, wherein the biomolecule is a nucleic acid.

68. The process of any one of claims 63-67, wherein the selected analog compound is an inhibitor of an activity of the biomolecule.

69. A compound that binds to a biomolecule, the compound selected using the method of any one of claims 1-62 or the process of any one of claims 63-68.
